(19) United States
(12) Patent Application Publication
(10) Pub. No.: US 2026/0309668 A1
(43) Pub. Date: Oct. 8, 2026

(54) ELECTROCHEMICAL METHOD AND SENSOR FOR PFAS DETECTION

(71) Applicant: B.G. NEGEV TECHNOLOGIES AND APPLICATIONS LTD., AT BEN-GURION UNIVERSITY, Beer-Sheva (IL)

(72) Inventors: Hadar Shmuel BEN YOAV, Beer Sheva (IL); Miriam AMIRAM, Beer Sheva (IL); Avner RONEN, Beer Sheva (IL); Meghna KHADKA, Beer Sheva (IL)

(21) Appl. No.: 19/483,418

(22) PCT Filed: May 16, 2024
(86) PCT No.: PCT/IL2024/050478

§ 371 Date: Nov. 11, 2025

Related U.S. Application Data
(60) Provisional application No. 63/466,737, filed on May 16, 2023.
(60) Provisional application No. 63/623,826, filed on Jan. 23, 2024.

(51) Int. Cl. G01N 27/30; G01N 33/18

(52) U.S. Cl. CPC G01N 27/30; G01N 33/1826

(57) ABSTRACT

A method for detecting and optionally quantifying one or more poly- and perfluoroalkyl substances (PEAS) in an aqueous sample, comprising adding a redox couple to the sample, electrochemically analyzing the sample with at least one surface-modified working electrode having fluorinated intrinsically disordered polypeptide deposited on its surface, by measuring current, voltage and/or calculating impedance as a PFAS-related signal, and determining the presence and optionally the level of PFAS, in the sample. An electrochemical sensor, a process of fabricating the sensor and an electrochemical sensing system comprising the sensor are also provided.

Poly- and perfluoroalkyl substances (PFAS) are widely used in many industrial sectors and can be found in a variety of commercial products, e.g., in non-stick cookware (Teflon® coatings), food packaging, raincoats, fire extinguishing foams and formulation aids (anionic surfactants) in agrochemical products. Perhaps the major PFAS surfactants are perfluorooctanoic acid (PFOA) and perfluorooctane sulfonic acid (PFOS) represented by the formulas shown below:

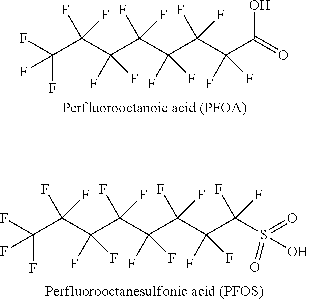 [SMILES: O=C(O)C(F)(F)C(F)(F)C(F)(F)C(F)(F)C(F)(F)C(F)(F)C(F)(F)F ; O=S(=O)(O)C(F)(F)C(F)(F)C(F)(F)C(F)(F)C(F)(F)C(F)(F)C(F)(F)C(F)(F)F]

PFAS have harmful and long-lasting effects on the environment, and can build up inside the human body, leading to increased cholesterol, diabetes, and kidney cancer. The U.S. Environmental Protection Agency recommends a 70 ppt concentration limit for PFOA and PFOS (PFAS Fact Sheets, 2024), and therefore high sensitivity and selectivity are needed for determination of PFAS in various environmental matrices. The standard techniques for detecting PFAS, which include gas or liquid chromatography and mass spectrometry, albeit fairly accurate, require advanced instrumentation facilities, with samples to be sent to a lab for analysis.

Thus, there exist a need for effective monitoring of PFAS in a field-deployable manner, i.e., a compact sensing device amenable to onsite detection of PFAS in potentially contaminated bodies of water, groundwater, and industrial wastewaters. Electrochemistry often lends onsite itself to applications, but the electrochemical detection and quantitative analysis of different PFAS in water and wastewater, bearing in mind the regulatory requirements, is not easy to meet.

Electrochemical detectability of PFAS depends on several factors, including an effective surface modification of the working electrode, that can respond to the presence of varying levels of PFAS in solution by creating an electrochemical signal in a concentration dependent manner, and a selection of an appropriate electrochemical technique for the measurement, showing acceptable limit of detection and sensitivity. A few approaches that were recently reported in the patent literature are presented below.

US 2023/0125201 shows immobilization of a redox indicator (Meldola Blue; MDB) by electrodeposition on an electrode surface. As the quaternary ammonium group of MDP interacts with the negatively charged functionality of PFOS, a change in the oxidation current of MDB occurs, measurable by voltammetry (differential pulse voltammetry).

US 2024/0035998 provides a sensing electrode with a coating formed by electrochemical polymerization of a monomer and the analyte (PFAS) on the electrode surface, followed by removal of the analyte from the coating, thus leaving cavities behind, which during testing, can be filled with, or occupied by, PFAS in the test solution. Differential pulse voltammetry was employed to characterize the response of the coated electrode to varying concentrations of PFOA in phosphate buffer saline in the presence of a redox mediator.

US 2024/0068971 relates to application of a polymer layer on the surface of the working electrode with affinity sites to bind (specifically or non-specifically) various PFAS. Voltammetry techniques (cyclic voltammetry and differential pulse voltammetry) were reported to detect PFOA and PFOS.

### THE INVENTION

Experimental results reported below show that PFAS can be detected electrochemically in an aqueous solution supplemented with a redox mediator, with the aid of an electrode coated with a thin film made of a fluorinated polypeptide (e.g., by electrochemical techniques such as voltammetry or even better, with electrochemical impedance spectroscopy).

For example, voltammograms recorded by cyclic voltammetry (CV), acquired with a working electrode coated with a fluorinated polypeptide, immersed in buffer solutions containing the ferrocyanide/ferricyanide redox couple [Fe(CN)₆³⁻↔Fe(CN)₆⁴⁻], show decreased oxidation and reduction peak currents in the presence of PFAS, compared with voltammograms generated by the bare electrode. With electrochemical impedance spectroscopy (EIS), a strong relationship was found between the concentration of PFAS and the charge transfer resistance (a parameter determined by EIS) for the fluorinated polypeptide-coated electrode (and other types of useful electrodes described below).

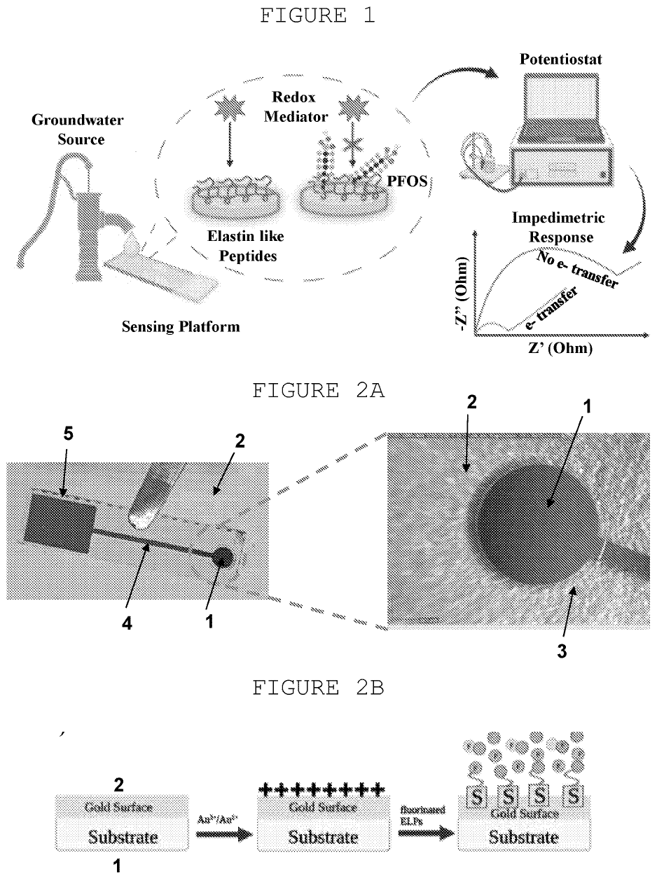

FIG. 1 shows the major components of the invention. A sample collected from the environment (e.g., groundwater sample) is contacted with a surface modified electrode. Effective surface modification was achieved by deposition of elastin like peptides (ELPs), as described in detail below. A redox couple (marked by the green star) is added to the solution, which is analyzed in an electrochemical cell connected to a potentiostat, to perform an electrochemical measurement with the surface modified electrode acting as the working electrode. In the presence of PFAS in the sample solution, the accessibility of the conducting electrode surface area to the redox pair is decreased, perhaps because the PFAS interacts with surface-deposited ELPs and affect the ELPs alignment on the electrode surface. EIS can sense the presence of PFAS in the solution.

Accordingly, the invention is primarily directed to a method for detecting and optionally quantifying one or more poly- and perfluoroalkyl substances (PFAS) in an aqueous sample, comprising adding a redox couple to the sample, electrochemically analyzing the sample with at least one surface-modified working electrode having fluorinated intrinsically disordered polypeptide deposited on its surface, by measuring current, voltage and/or calculating impedance as a PFAS-related signal, and determining the presence of PFAS and optionally the level of PFAS, in the sample.

Another aspect of the invention is an electrochemical sensor, comprising the fluorinated intrinsically disordered polypeptide-coated electrode as a working electrode. The fluorinated intrinsically disordered polypeptide-coated electrode is usually incorporated in an array of working electrodes, alongside at least one electrode selected from the group consisting of bare electrode and electrodes coated with non-fluorinated intrinsically disordered polypeptides showing varying degree of hydrophobicity (e.g., polypeptides consisting mostly of hydrophobic natural and/or unnatural amino acids versus mostly hydrophilic natural and/or unnatural amino acids, or controlling hydrophobicity by incorporation of modified amino acids bearing hydrophilic/hydrophobic substituents at specific positions).

The method of the invention can be used to determine quantitatively different PFAS. PFAS that can be detected are represented by the formulas R—CₙF₂ₙ₊₁ or R′—CₙF₂ₙ—R″ where n is in integer in the range from 3 to 15, e.g., from 5 to 15, and R, R′, R″ are functional groups devoid of carbon or C—F bonds, such as —COOH and —SO₃H. PFAS may be divided into long and short chain derivatives. Examples of long chain (n≥5) PFAS include PFOA and PFOS which were mentioned above and perfluorononanoic acid (PENA; R=COOH, n=8) and perfluorodecanoic acid (PFDA; R=COOH, n=9). As an example of short chain PFAS, perfluorobutanoic acid (PFBA; R=COOH, n=3) may be mentioned. In the experimental work reported below, PFOS was tested, either alone or in admixture with PFOA, PFHXS, PFHXA, PFBA and PFBS.

The sample may be collected from the environment (a waterbody, groundwater with up to 200 ppt PFAS), from industrial and municipal streams (wastewaters with up to 300 ppb PFAS and even higher, effluents for irrigation with up to 300 ppb PFAS) and is usually conducted onsite, in a field-deployable manner, with the aid of portable sensors the design of which is described below.

The working electrode is preferably made of a noble metal, e.g., gold, platinum, rhodium, and iridium. A carbon-based electrode (e.g., a glassy carbon electrode) can also be used. The sensor of the invention is not limited to specific design. One useful configuration is a chip configuration with one or more electrodes deployed on a substrate, e.g., a glass wafer, optionally atop of a titanium adhesion layer attached to the substrate. FIG. 2A shows an individual gold electrode (1), created by photolithography and lift-off techniques described below. The electrodes may be disc-shaped, with surface area in the range of 5 to 20 mm². The electrodes are protruding from the surface of the substrate (2), with each electrode being encircled by a wall made of an epoxy polymer (3). The electrode is joined by a thin wire (4) to a contact pad (5), to be connected to a potentiostat or a galvanostat.

Accordingly, another aspect of the invention is a process for preparing an electrochemical sensor by photolithography and lift-off techniques, comprising:
depositing on a substrate an electrode material, optionally atop of an adhesion layer (e.g., made of titanium) attached to the substrate, to create electrodes that are spaced apart from each other, the electrodes are protruding from the surface of the substrate, wherein each electrode is encircled by a wall made of a polymer (e.g., epoxy);
cleaning the electrode surface; and
coating at least one electrode with a fluorinated intrinsically disordered polypeptide, and optionally other electrodes with non-fluorinated polypeptides that are described in more detail below.

Coating and immobilization of the fluorinated intrinsically disordered polypeptide onto a noble metal can be achieved by incorporation of cysteine (N-terminal, C-terminal, internal) into the fluorinated polypeptide, to benefit from the strong interaction between the thiol (—SH) end group and the metal. Subsequently, application of a terminal cysteine-fluorinated polypeptide onto the electrode surface, by employing coating techniques such as drop-casting, spin-coating and spray coating, results in the deposition of a thin film consisting of the fluorinated polypeptide on the electrode surface.

The preferred method of coating is drop-casting, and such an approach is shown schematically in FIG. 2B. A solution of fluorinated intrinsically disordered polypeptide with a cysteine end group [in PBS, at concentration from 15 to 50 μM] is spread over a noble metal, e.g., gold electrode (1) patterned on the SiO₂ wafer. To coat a surface area of 5 to 20 mm², typical of electrodes for use in this invention, the volume of the coating solution is from 1 to 10 μL. FIG. 2B shows the gold-sulfur coupling. The fluorine-containing functional groups embedded in the polypeptide applied on the gold surface (e.g., fluorinated alkyl such as trifluoromethyl, —CF₃) are labeled by circles with the letter F inside. Then the film is dried under controlled conditions, e.g., incubation for a few hours (from 1 to 12 h) at 4° degrees or at room temperature, achieving stable immobilization of the polypeptides on the electrode surface in the form of thin solid films. Lastly, the film is washed with PBS and DDW (whereby presumably a monolayer is formed).

It should be noted that after the fabrication of the electrode on the SiO₂ substrate, and prior to the coating step, the chip is cleaned, e.g., with acetone, methanol, isopropanol, followed by washing with DDW to remove the organic (photoresist) residues from the top of the electrodes. Next, the electrode is cleaned to improve electrochemical detectability. Several cleaning protocols can be used for this purpose, for example, treatment with ultraviolet ozone, sulfuric acid and potential cycling, aqua regia (nitric acid/hydrochloric acid), piranha solution (sulfuric acid+hydrogen peroxide) and potassium hydroxide+hydrogen peroxide solution. The latter technique (immersing the chip in H₂O₂/KOH solution (for example, 30% hydrogen peroxide and 0.3 M of KOH solution combined at 3:1 to 7:1 volumetric ratio) for not less than five minutes, e.g., ~ ten minutes, with subsequent drying, e.g., by application of nitrogen dispensed from a spray gun, was shown to enable effective immobilization of the polypeptide on the electrode. Perhaps the KOH/H₂O₂ treatment activates the gold surface in a manner increasing its affinity towards the polypeptide. X-Ray Photoelectron Spectroscopy (XPS) analysis reported below detected the presence of C, N and O on the electrode surface, attesting to the creation of a drop-casted film made of the polypeptide onto a surface of gold. Accordingly, the invention provides a process for preparing a sensor as defined above, wherein the cleaning step comprises treating the electrode with a solution of alkali (e.g., potassium) hydroxide and hydrogen peroxide.

As pointed out above, carbon-based electrodes (e.g., glassy carbon) can also be used, in which case immobilization of the polypeptides onto the surface requires other type of linkages, so as to strongly bind the polypeptides chains to the surface. Acceptable surface attachment chemistries are detailed for example in (https://doi.org/10.1142/S1793048006000045, https://doi.org/10.1007/s00604-015-1623-4)

The arrays of working electrodes, for example, with the following composition of bare and coated electrodes:
n_{fluorinated polypeptide}, n_{bare}, n_{non-fluorinated polypeptide}

(n, the number of electrodes of each type, may be independently a number from 1 to 5; n>1 for repetitions), may be arranged in different designs and geometries, including a simple and straightforward configuration based on the use of an electrochemical measurement cup to hold the sample, fitted with a perforated cover; the holes in the cover correspond in number and size to the electrodes, such that individual working electrodes can be inserted into the measurement cup through the holes to be immersed in the sample. Commercial counter electrode (e.g., commercial Pt wire) and commercial reference electrode (Ag/AgCl) are also inserted into the cup.

A more sophisticated design is the one shown in WO 2018/225058. e.g., a cylindrical body made of silicon, polyvinyl alcohol or polydimethylsiloxane, which is a few cm long and with diameter is in the range from 2 to 3 cm. The accessible surfaces of the electrodes were deployed on one base of the tubular body: a disc-shaped reference electrode positioned concentrically and coaxially in respect to the cylindrical body, a counter electrode at the vicinity of the reference electrode and multiple surface modified working electrodes positioned in radial direction from the reference and counter electrodes and evenly distributed along the perimeter of the base of the cylindrical body.

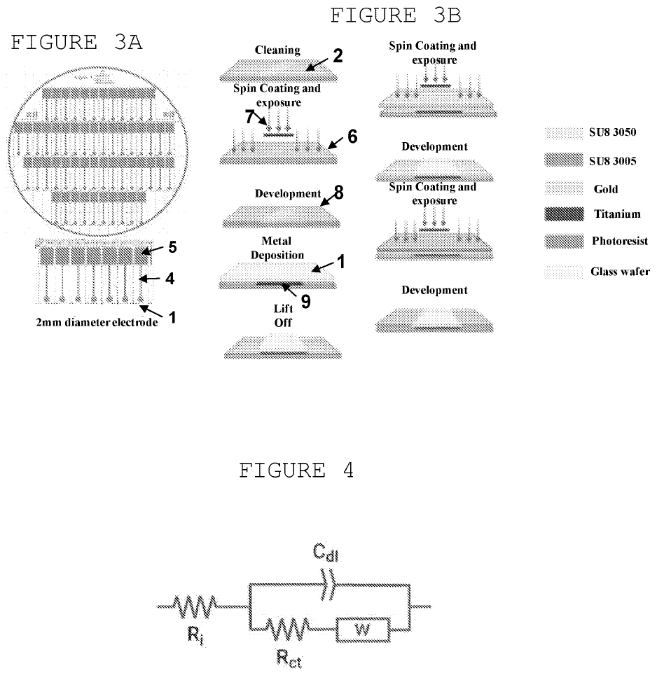

Alternative designs are based on fabrication of electrodes on silicon wafers using lithography and liftoff techniques as mentioned above in reference to FIG. 2A and shown in more detail in reference to FIG. 3, e.g., for a portable device. Briefly, a substrate (2) is cleaned, a first photoresist (6) is applied (either negative, positive or image reversal resist), e.g., by spin coating, spray coating or dip coating, to produce a thin uniform layer on the substrate, followed by soft baking. A first mask is aligned, to transfer the pattern corresponding to electrodes' sites onto the surface of the substrate. The photoresist is exposed through the pattern on the mask with UV light (7) followed by a development step (8). Next, bare electrodes are deposited in the intended sites, e.g., first titanium which serves as an adhesion layer (9) and then gold (1) followed by lift off procedure that results in a plurality of individual, spaced apart, gold electrodes protruding from the glass substrate. The gold electrodes need to be encircled by a wall, to define the active surface area of the electrode. This is accomplished again by photolithography, as shown by the sequence of steps depicted in the right column of FIG. 3B. The gold electrode is encircled by a wall made of epoxy polymer, e.g., bisphenol A Novolac epoxy such as SU-8 photoresist, which is 5-7 μm thick.

FIGS. 3A-3B show just one approach to the structuring of electrodes on a wafer. The application of positive, negative or image reversal photoresists is often a matter of choice and convenience. Also, structuring via etching (i.e., wet etching/dry is also possible.

Having patterned the electrodes on the substrate, the desired coatings are applied, e.g., by drop-casting as described above. In operation, the electrochemical sensor of the invention may be combined with commercial counter and reference electrodes to be connected to a potentiostat or galvanostat to run the electroanalytical method, e.g., voltammetry or EIS. However, a disc-shaped counter microelectrode, and Ag/AgCl electroplated reference microelectrode may be applied on the base substrate adjacent to the working electrode. Silver/silver chloride miniature electrodes are prepared as described in WO 2022/137238, Example 6.

Turning now to the fluorinated and non-fluorinated polypeptides that are used to modify the surface of the electrodes, in their most general form, these are intrinsically disordered proteins such as elastin-like polypeptides (ELPS), resilin-like polypeptides, or other artificial polypeptides such as Proline/alanine-rich sequence (PAS) and the unstructured polypeptide Xten, that may or may not demonstrate temperature responsive phase transition behavior, having fluorinated residues (and corresponding non-fluorinated polypeptides).

Especially useful are ELPs comprising a repeat unit having the amino acid sequence VPGXG (Val-Pro-Gly-X-Gly denoted by SEQ ID NO. 1, i.e., the ‘VPGXG’ pentapeptide motif) wherein X, termed the guest-residue position, is any natural amino acid (except proline) or synthetically modified amino acid (also known as “unnatural” or “non-canonical amino acid”, ncAAs). The number of repeat units (i.e., the pentapeptide motif) is at least 15, e.g., from 15 to 240, e.g., from 30 to 150, e.g., from 30 to 100.

For example, the fluorinated and non-fluorinated polypeptides are ELPs comprising a repeat unit having the amino acid sequence VPGXG, wherein X is any natural amino acid, apart from proline, or unnatural i.e., modified amino acid, characterized in that:
the fluorinated polypeptide (fluorinated ELP) comprises from 5 to 100%, e.g., from 5 to 50%, e.g., from 10 to 25%, a repeat unit having the amino acid sequence VPGXG, wherein X=X¹ which is a modified amino acid bearing a fluorinated functionality, such as the repeat unit having the amino acid sequence denoted by SEQ ID NO. 2, comprising p-Trifluoromethyl-L-phenylalanine at the guest-residue position (percentage is calculated relative to the total number of VPGXG repeat units);
a first non-fluorinated polypeptide which comprises from 5 to 100%, e.g., from 5 to 50%, e.g., from 10 to 25%, a repeat unit having the amino acid sequence VPGXG, wherein X=X² which is a natural or unnatural amino acid bearing a hydrophilic group, such as —OH moiety, e.g., the repeat unit having the amino acid sequence VPGYG denoted by SEQ ID NO. 8, where tyrosine is incorporated at the guest-residue position (calculated relative to the total number of VPGXG repeat units); and
a second non-fluorinated polypeptide which comprises from 5 to 100%, e.g., from 5 to 50%, e.g., from 10 to 25%, a repeat unit having the amino acid sequence VPGXG, wherein X=X³ is a modified amino acid bearing a hydrophobic moiety, such as the repeat unit having the amino acid sequence denoted by SEQ ID NO. 3, comprising 4-propargyloxy-L-phenylalanine at the guest-residue position (calculated relative to the total number of VPGXG repeat units).

Exemplary fluorinated modified (synthetic) amino acid residues which can serve as X¹ units are derived from the natural amino acid residues Tyrosine (Tyr, Y), Phenylalanine (Phe, F), Tryptophan (Trp, W), Alanine (Ala, A), Valine (Val, V), Isoleucine (Ile, I), Leucine (Leu, L) and Methionine (Met, M). Preferably, the fluorinated hydrophobic modified (synthetic) amino acid residues are derived from aromatic amino acid residues, i.e., from Phe, Tyr or Trp. Fluorinated functionality is one (or more) fluorine atoms covalently bonded to, e.g., a carbon ring, or fluorinated alkyl such as C1-C5 alkyl, covalently bonded to a carbon ring. A sufficient level of fluorine atoms in the polypeptide is not less than 5 atoms per polypeptide, e.g., not less than 10, e.g., from 15 to 45, e.g., from 25 to 35; or from 0.1 to 0.5% by weight.

For example, X¹ is L-phenylalanine substituted at positions ortho (o), meta (m), or para (p) with fluorine atom(s) or fluorinated C1-C5 alkyl(s), X² is 4-hydroxy-L-phenylalanine (tyrosine) and X³ is L-phenylalanine substituted at positions o, m, or p with an C1-C5 alkoxy, alkenyloxy or alkynyloxy group.

Preferably, X¹ is trifluoromethyl-substituted L-phenylalanine and X³ is alkynyloxy (e.g., propargyloxy)-substituted L-phenylalanine, e.g. X¹ and X³ are:

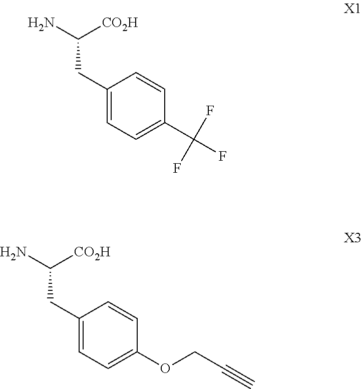 [SMILES: N[C@@H](Cc1ccc(C(F)(F)F)cc1)C(=O)O ; C#CCOc1ccc(C[C@H](N)C(=O)O)cc1]

In addition to p-trifluoromethyl-L-phenylalanine that is depicted above, the following compounds can be incorporated into the the fluorinated unit: polypeptides as m-trifluoromethyl-L-phenylalanine, m-fluoro-L-phenylalanine, p-fluoro-L-phenylalanine, 2,6-difluoro-L-phenylalanine, 2,4-difluoro-L-phenylalanine, 3,4,5-trifluoro-L-phenylalanine, 2-fluoro-L-phenylalanine and 3-fluoro-L-tyrosine, all of which are commercially available (e.g., from Chemimpex).

More specifically, the fluorinated polypeptide exemplified herein comprises amino acid sequence shown in SEQ ID. 6, the first non-fluorinated polypeptide has the sequence shown in SEQ ID. 5 and the second non-fluorinated comprises amino acid sequence in SEQ ID. 7.

The polypeptides for use in the present disclosure may be prepared by any method known in the art, according to the desired amino acid sequence. In particular, ELPS may be prepared by bioengineering methods known to the skilled artisan, which include inserting a nucleic acid sequence encoding the polypeptide into a suitable host (e.g., a bacterial strain), expressing the encoded polypeptide and purifying it from the resulting culture. By way of example, when the polypeptide comprises modified amino acid residues, the polypeptide may be prepared by following the procedures described by Gueta, O. et al., (Frontiers in Bioengineering and Biotechnology, 2022, Volume 10, Article 913057) and in the published international application WO 2021/165968, both of which are incorporated herein by reference. In general, the above preparation methods for ELPs comprising modified amino acid residues involve the incorporation of specific modified amino acid residues using specialized, engineered aminoacyl tRNA synthetases (aaRSs) with different substrate specificities, thereby enabling multi-site incorporation of different modified amino acid residue, based on the specificity of the aaRSs being used.

The polypeptides for use in the present disclosure may further comprise additional amino acid sequences, optionally modified amino acids, such as short linkers positioned internally or at the N terminus or the C terminus of the polypeptide.

Prior to the electrochemical analysis, the test sample is supplemented with a redox couple, e.g., at a concentration in the range from 1 mM to 10 mM. Suitable redox couples include the ferrocyanide/ferricyanide couple and other iron-based couples, e.g., ferrocene carboxylic acid/FCA⁻.

Then the electrochemical cell is assembled by connecting the electrodes to e.g., potentiostat, optionally with the aid of a multiplexer, to run the electrochemical analysis, such as voltammetry where a potential sweep takes place and the current is measured as the analytic signal (cyclic voltammetry (CV), or differential pulse voltammetry (DPV)); or electrochemical impedance spectroscopy (EIS) where a sinusoidal potential over a frequency range is applied and current is measured, and resistance and/or capacitance are usually calculated. FIG. 4 shows the Randles equivalent circuit that was used for fitting the impedance spectra produced by the EIS, i.e., the Nyquist and Bode plots. The Randles circuit of FIG. 4 is a series combination of the solution resistance Rᵢ, with the double layer capacitance C_{dl}, which is in parallel with the charge transfer resistance R_{ct} (associated with the faradaic reaction), which is in series with the Warburg impedance W.

Accordingly, the invention provides a method of quantifying one or more poly- and perfluoroalkyl substances (PFAS) in an aqueous sample with the aid of the electrodes described above, comprising analyzing the sample by EIS to generate impedance spectra, fitting the impedance spectra with Randles circuit, and calculating one or more of the following components of the Randles circuit: the charge transfer resistance, the solution resistance, the double layer capacitance and the Warburg impedance. As shown below, the charge transfer resistance correlates strongly with the concentration of the PFAS in solutions.

As to the chemometric model, a Partial Linear Regression Model (PLSR) can be used to determine the PFAS concentration. To this end, the Origin Pro program can be used to organize the Bode plot data into the cell format. In the matrix made out of specific array sensors, the raw impedance spectra are assembled. Sequential placement of the signals was done for individual sensors. The approach used was partial least square regression (PLSR), which is a combination of Principal Component Analysis (PCA) and Multiple Linear Regression (MLR). The statistical analysis was aided with Origin Pro 2024 Software where the statistics panel was selected, under the Multivariate Analysis (Partial Least Squares) is provided. It performs effectively when there is limited sample availability and a significant connection between many attributes. The independent variable chosen is the impedance values at varying frequencies. The dependent variable chosen is the analyte solution's concentration. For prediction, the identified independent variables were used. The components were extracted using the Singular Value Decomposition (SVD) technique of computation. A cross-validation procedure is then used to confirm the anticipated model. Then, using Origin Pro software, the ideal number of components is calculated using the predicted residual sum of squares (PRESS) and its root mean. The number of factors involved is known as the “optimal number of factors” when the smallest root mean is attained. Hence, the quality of the model is then assessed by the various combination of root mean PRESS between the actual concentration and predicted concentration. The efficiency of the model was further evaluated by calculating (limit of detection) LOD, Pearson's Co-relation Co-efficient (PCC), and PRESS values.

Accordingly, the invention further provides a method comprising analyzing the sample by EIS to generate impedance spectra and using a Partial Linear Regression Model (PLSR) to determine the PFA concentration based on the measured impedance spectra, wherein the PLSR model was created using as independent variable impedance values at varying frequencies, and the dependent variable is the PFAS concentration in solution.

Model predictability was assessed by testing with unseen samples. Good results were obtained with the combinations of electrodes consisting of:
the fluorinated polypeptide-coated electrode, wherein the fluorinated polypeptide (fluorinated ELP) comprises from 5 to 100%, e.g., from 5 to 50% (10 to 25%), a repeat unit having the amino acid sequence VPGXG, wherein X=X¹ which is a modified amino acid bearing a fluorinated functionality, e.g., the repeat unit has the amino acid sequence denoted by SEQ ID NO. 2, comprising p-Trifluoromethyl-L-phenylalanine at the guest-residue position (percentage is calculated relative to the total number of VPGXG repeat units);
   at least one bare electrode; and
   at least one electrode selected from:
   a first non-fluorinated polypeptide which comprises from 5 to 100%, e.g., from 5 to 50%, (10 to 25%), a repeat unit having the amino acid sequence VPGXG, wherein X=X² which is a natural or unnatural amino acid bearing a hydrophilic group, e.g., —OH, such as the repeat unit of the amino acid sequence denoted by SEQ ID NO. 8, where tyrosine is incorporated at the guest-residue position (calculated relative to the total number of VPGXG repeat units); and
   a second non-fluorinated polypeptide which comprises from 5 to 100%, e.g., from 5 to 50%, (10 to 25%), a repeat unit having the amino acid sequence VPGXG, wherein X=X³ is a modified amino acid bearing a hydrophobic moiety, such as the repeat unit having the amino acid sequence denoted by SEQ ID NO. 3, comprising 4-propargyloxy-L-phenylalanine at the guest-residue position (calculated relative to the total number of VPGXG repeat units), allowing quantification of PFAS in a concentration range of 5 nM to 500 nM with LOD as low as 1-2 nM. Additional aspect of the invention is an electrochemical sensing system comprising the sensor described above, with the working electrodes, counter electrode and reference electrode connected to a potentiostat or a galvanostat, and a processor configured to determine, based on a chemometric model, the concentration of PFAS in a sample.

### EXAMPLES

### Materials

Acetone, isopropanol, methanol, hydrochloric acid, sulfuric acid and hydrogen peroxide were used without further purification. Simulated ground water was prepared using the Humic acid (Thermo Fisher Alfa Aesar), Potassium Chloride (KCl) from Acros Organics and Sodium Sulphate (Na₂SO₄) from Alfa Aesar, Deionized water (>18 MΩ) was obtained from a Super Q water system (Millipore). For all electrochemical characterization, the redox couple 5 mM ferrocyanide/ferrocyanide (Fisher Scientific) dissolved in 10 mM phosphate buffer saline (PBS) solution prepared from PBS tablet (Acros Organics) was used.

### Methods

### Electrochemical Measurements

All electrochemical measurements were performed on a VSP potentiostat (Bio-Logic Ltd); a three-electrode system consists of a working gold/surface modified gold electrode (2 mm diameter), a commercial Pt wire as a counter electrode (CHI115, CH Instruments) and Ag/AgCl (011464, BAS Inc.) coated Tungsten needle (P/N H-20242, Quarter) employed as a reference electrode. The potential values presented in this manuscript are all with reference (Ref) to an Ag/AgCl half-cell potential.

### Statistical and Chemometric Analysis

The statistical and chemometric analysis of the electrochemical data were analyzed using the Origin (Pro 2024) software. The sensitivity via the slope and the limit of detection via the intercept were extracted from the linear regression curve plotted in Origin (Pro 2024) software. The multivariate analysis namely Principal Component Analysis and Partial Least Square Regression were also evaluated in Origin (Pro 2024) software. The hypothesis statistical test was performed by Welsch's ANOVA test app. The Bode plot data, which includes impedance vs frequency and phase vs frequency were used to perform the multivariate analysis.

### Preparation 1

### Preparation of PFOS Samples

### Buffer Solutions

To prepare the stock 10× Phosphate buffer (PBS) solution (pH 7.4), one PBS tablet was dissolved in 200 ml of DDW. Further, 5 mM ferrocyanide/ferricyanide solution (the redox mediator) was prepared with equimolar ratio of ferrocyanide and ferricyanide in PBS solution. A stock solution of 100 ppm PFOS was prepared in PBS containing ferrocyanide/ferricyanide which is stored at room temperature. PFOS standard solutions of concentration ranging from 500 nM to 5 nM were prepared by diluting the stock solution of 100 ppm PFOS samples with 5 mM ferrocyanide/ferricyanide dissolved in PBS solution. (10×, pH 7.4).

### Simulated Ground Water Solutions

The simulated ground water was prepared by adding salts 5 mM (KCl, Na₂SO₄), 10 uM humic acid and ferrocyanide/ferricyanide in DDW under constant stirring on a magnetic stirrer. The concentration of the salts and humic acid used to prepare the simulated solution is similar to the concentration of salts existing in real ground water samples.

### Real Groundwater Samples

The real groundwater samples were collected from the groundwater drill located near LoD, Israel. The samples were collected in a 50 mL falcon tubes and stored at room temperature. For the electrochemical analysis of PFOS, the real groundwater samples were spiked with PFOS and further diluted at various concentration ranging from 5 nM to 500 nM along with the redox mediator.

### Preparation 2

### Electrode Fabrication on a Glass Substrate

An individual disc-shaped gold electrode deposited on a silicon wafer is shown in FIG. 2A, with diameter of 2 mm, encircled by SU8-3005 wall which is 5-7 μm thick. Individual gold electrode chips like the one shown in FIG. 2A were prepared using photolithography and lift-off techniques described below.

The wafer was cleaned with acetone, isopropanol, and N₂-gun. The wafer was dehydrated at a hot plate temperature of 110° C. for a period of 10 minutes. The spin coating included spinning at sequential steps, starting from spinning at 500 rpm with acceleration rate of 100 rpm/s for 20 seconds, following through spinning at 3000 rpm with acceleration rate of 300 rpm/s for 40 seconds and finally spinning at zero rpm with acceleration rate of 100 rpm/s for 1 second using a four-inch holder.

7 milliliters of photoreactive chemicals are positioned in the center of wafer using a pipette before spinning begins. The process of spin coating should encompass about three-quarters of the entire wafer and while this is happening, the temperature of hot plate is checked against power supply for lamp at 30 degrees Celsius. Then prebake wafers at 110 degrees Celsius for 60 seconds before transferring them to mask aligner to do exposure and alignment steps. After that transfer wafer into mask aligner and set exposure parameters. Afterwards bake again at 110 degrees Celsius for another minute when development step comes in where the wafer is put in MIF 726 Developer for one hundred ten seconds and rotated gently so as to ensure uniformity.

Next, the deposition of Ti/Au 20/200 nm using E-beam VST process takes place. The NMP solution was added into the glass bowl. For complete wafer coverage by warm NMP, the bowl was agitated. The wafer was dipped in warm NMP for about 20 minutes before removing it away. Then the wafer is rinsed with isopropanol and DDW respectively and finally dry it using N₂ gun. Quality of lift-off process is done under a microscope. Start cleaning the wafer by applying Acetone, Isopropanol and N2-Gun to clean it thoroughly.

Then the wafer is dehydrated at 95 degrees for 5 minutes on a hot plate. The mask aligner should meanwhile be switched off as well as warmed up for five minutes. Thereafter, the hot plate is activated during this waiting interval and set its temperature to 150 degrees, and spinning protocol is repeated as mentioned earlier. Once the wafer is placed on the spinner the vacuum is started. 7 ml of photoresist was added to the center of the wafer then spun it for 5 minutes. At the time the plate was switched on and raised the temperature to 95 degrees. Next baking the wafer was done at 95 degrees for 8 minutes. The wafer was placed in the mask aligner and wait for around 5 minutes to ensure that the hot plate reaches a temperature of 95 degrees. The other plate was heated to the temperature up to 150 degrees. Conduct an exposure bake at 95 degrees for 4 minutes. Moving on to the development phase glass bowls and wafer holders were prepared for use. The glass bowl was filled with AZ Z EBR liquid and another with Isopropanol. The wafer was placed in the AZ EBR glass bowl for 6 minutes rotating it gently. The wafer is rinsed in the Isopropanol bowl for 10 seconds followed by cleaning with DDW and N2-Gun. To complete the process, a hard bake is performed by baking the wafer at 150 degrees for 5 minutes. The process quality was evaluated under the microscope and profilometer.

Prior to coating with the ELPs as described below, the chips were cleaned by rinsing with acetone, methanol, isopropanol, followed by washing with DDW to remove the organic (photoresist) residues from the top of the electrodes. Next, the chip was immersed in KOH/H₂O₂ solution (16 ml hydrogen peroxide, 3.2 ml of KOH) for ten minutes, and finally dried using a nitrogen gun.

### Preparation 3

### ELP Deposition Solutions

Three types of Elastin like peptides (ELPs) were prepared for deposition on the electrodes.

A non-fluorinated ELP, named ELPs-tyr, has the amino acid sequence denoted by SEQ ID NO. 5:

GVPGGGVPGAGVPGGGVPGAGVPGGGVPGYGVPGGGVPGAGVPGG
GVPGAGVPGGGVPGYGVPGGGVPGAGVPGGGVPGAGVPGGGVPGY
GVPGGGVPGAGVPGGGVPGAGVPGGGVPGYGVPGGGVPGAGVPGG
GVPGAGVPGGGVPG.YGVPGGGVPGAGVPGGGVPGAGVPGGGVPG
YGVPGGGVPGAGVPGGGVPGAGVPGGGVPGYGVPGGGVPGAGVPG
GGVPGAGVPGGGVPGYGVPGGGVPGAGVPGGGVPGAGVPGGGVPG
YGVPGGGVPGAGVPGGGVPGAGVPGGGVPGYGGGC.

Other ELPs have the same amino acid sequence as ELPs-tyr, however, their amino acid sequences include the non-canonical amino acid residues p-Trifluoromethyl-L-phenylalanine (CAS No. 114926-38-4, Chemimpex) or 4-propargyloxy-L-phenylalanine (CAS No. 1080496-42-9, Chemimpex) at each one of the positions of the amino acid residues Tyrosine (Y) at the sequence denoted by SEQ ID NO. 5, resulting in ELPs termed, respectively, fluorinated ELPs (ELPs-tFF) and non-fluorinated ELP (ELPs-pPr).

The amino acid sequence of the fluorinated ELPs (ELPs-tFF):

GVPGGGVPGAGVPGGGVPGAGVPGGGVPGtFFGVPGGGVPGAGVP
GGGVPGAGVPGGGVPGtFFGVPGGGVPGAGVPGGGVPGAGVPGGG
VPGtFFGVPGGGVPGAGVPGGGVPGAGVPGGGVPGtFFGVPGGGV
PGAGVPGGGVPGAGVPGGGVPGtFFGVPGGGVPGAGVPGGGVPGA
GVPGGGVPGtFFGVPGGGVPGAGVPGGGVPGAGVPGGGVPGtFFG
VPGGGVPGAGVPGGGVPGAGVPGGGVPGtFFGVPGGGVPGAGVPG
GGVPGAGVPGGGVPGtFFGVPGGGVPGAGVPGGGVPGAGVPGGGV
PGtFFGGGC.

The term tFF indicates the positions of p-Trifluoromethyl-L-phenylalanine in the amino acid sequence, which is denoted herein by SEQ ID NO. 6.

The amino acid sequence of the hydrophobic ELPs (ELPs-pPr):

GVPGGGVPGAGVPGGGVPGAGVPGGGVPGpPrGVPGGGVPGAGVP
GGGVPGAGVPGGGVPGpPrGVPGGGVPGAGVPGGGVPGAGVPGGG
VPGpPrGVPGGGVPGAGVPGGGVPGAGVPGGGVPGpPrGVPGGGV
PGAGVPGGGVPGAGVPGGGVPGpPrGVPGGGVPGAGVPGGGVPGA
GVPGGGVPGpPrGVPGGGVPGAGVPGGGVPGAGVPGGGVPGpPrG
VPGGGVPGAGVPGGGVPGAGVPGGGVPGpPrGVPGGGVPGAGVPG
GGVPGAGVPGGGVPGpPrGVPGGGVPGAGVPGGGVPGAGVPGGGV
PGpPrGGGC.

The term pPr indicates the positions of 4-propargyloxy-L-phenylalanine in the amino acid sequence, which is denoted herein by SEQ ID NO. 7.

The three ELPs described above were expressed and purified by following the procedures described by Gueta, O. et al., (Frontiers in Bioengineering and Biotechnology, 2022, Volume 10, Article 913057) and in the published international application Wo 2021/165968, both of which are incorporated herein by reference. In general, the preparation of ELPs comprising ncAAs described by Gueta, O. et al. relies on the incorporation of ncAAs using engineered aminoacyl tRNA synthetases (aaRSs) with specific substrate specificities that enable an efficient multi-site incorporation of different ncAAs, based on the specificity of the aaRSs used and the availability of the relevant ncAAs in culture medium during translation.

Briefly, E. coli strains (the genomically recoded C321.ΔA strain, Addgene #73,581, which is used for efficient multi-site incorporation of multiple uAAs per protein), harboring plasmids encoding the aminoacyl tRNA synthetases M. jannaschii tyrosyl-tRNA synthetase (MjTyrRS, which is the native aaRSs) for tyrosine incorporation, Mut1-RS for pPR incorporation, or the pAcFRS.2.t1 for tFF incorporation and the ELP polypeptide (the ELP polypeptide was encoded by the nucleic acid sequence denoted by SEQ ID NO. 4) were used as starter cultures (1:40 v/v of final expression volume) in 2×YT media, supplemented with kanamycin (30 μg ml⁻¹) and chloramphenicol (25 μg ml⁻¹). The cultures were incubated overnight at 34° C. while shaking at 220 rpm, and transferred to expression flasks containing 2×YT media, antibiotics, arabinose (0.2%), and 1 mM of the corresponding ncAA (i.e., pPR was added in the case of Mut1-RS and tFF in the case of pAcFRS.2.t1. In the case of native MjTyrRS, no ncAA was added, as the enzyme uses tyrosine available in the growth media. Plasmids encoding the aminoacyl tRNA synthetases and the ncAAs detailed above are commercially available, e.g., plasmids can be obtained from Addgene and ncAAs from Chem-impax.

Cells were allowed to grow at 34° C. while shaking. At an optical density of 0.5-0.8 at 600 nm (OD₆₀₀) protein expression was induced with isopropyl β-d-1-thiogalactopyranoside (IPTG, 1 mM) and incubation continued under the above conditions until harvest.

The cells were harvested 24 hours after inoculation by centrifugation at 4,000 g for 30 minutes at 4° C. The cell pellet was then resuspended by vortex in milli-Q water (~4 ml) and either stored at −80° C. or purified immediately. Protein purification was performed via a process known as inverse-transition cycling, in which phase separation of ELPs is induced via heat or the addition of salt, followed by centrifugation to separate the ELP pellet. Then, the pellet is re-dissolved in cold water or buffer, and irreversibly precipitated E. coli protein contaminants are centrifuged and removed. This process is repeated 3-5 times until the ELP protein is sufficiently pure. For purification, resuspended pellets were lysed by ultrasonic disruption (18 cycles of 10 seconds sonication, separated by 40 seconds intervals of rest). Poly (ethyleneimine) was added (0.2 ml of a 10% solution) to each lysed suspension before centrifugation at 4,000 rpm for 15 minutes at 4° C. to separate cell debris from the soluble cell lysate containing the polypeptides. All ELP constructs were purified by a modified inverse transition cycling (ITC) protocol (Hassouneh et al., 2010) consisting of multiple “hot” and “cold” spins by using sodium chloride to trigger the phase transition. Before purification, the soluble cell lysate was incubated for up to 2 minutes at 42-65° C. to denature the native E. coli proteins. The cell lysate was then cooled on ice, centrifuged for 2 min at ~14,000 rpm, and the pellet was discarded. For “hot” spins, the ELP phase transition was triggered by adding sodium chloride to the cell lysate (from which the native E. coli proteins pellet was discarded) or to the product of a previous cycle of ITC at a final concentration of up to ~5 M. The solutions were then centrifuged at ~14,000 rpm for 10 minutes and the pellets were resuspended in milli-Q water, after which a 2 minutes “cold” spin was performed without sodium chloride to remove denatured contaminants. Additional rounds of ITC were conducted as needed using a saturated solution of sodium chloride until sufficient purification was achieved. Purified proteins were visualized on SDS-PAGE.

### Example 1

### Immobilization of ELPs on Gold Electrodes and Characterization of the Modified Electrodes

### Preparation

The ELPs solutions of Preparation 3 were diluted to 25 μM in 10 mM PBS solution (pH=7.4). Thereafter, the 25 μM ELPs solutions namely, ELPs-tyr, ELPs-tFF, ELPs-pPr were immobilized on the gold macroelectrode via drop cast method followed by incubation for two hours at 40 degrees, achieving stable immobilization of the ELP on the electrode surface in the form of thin solid films. Next, the films were washed with PBS and DDW.

### Surface Characterization of the ELPs-Modified Electrodes

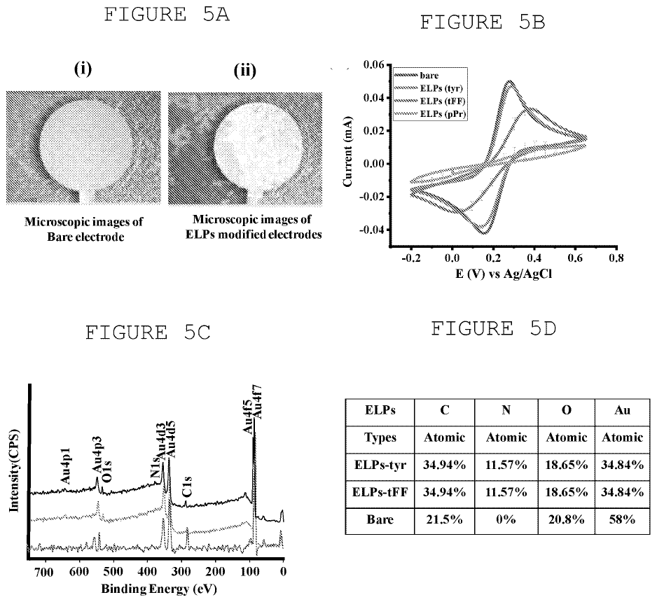

The microscopic images for a bare and an ELPs modified electrode (the fluorinated polypeptide-coated electrode) were taken by optical microscopy (MX-50A, Olympus), and are shown in FIG. 5A (i) and (ii), respectively. The images show a difference between the surface of the bare gold electrode and ELPs film-coated gold electrode.

The elemental analysis was performed by X-Ray Photoelectron Spectroscopy (XPS). (ESCALAB Xi+ Thermo-Fisher Scientific). The XPS analysis shown in FIG. 5C and the corresponding data tabulated in FIG. 5D attest to the successful immobilization of the ELPs on the gold macroelectrode: the elemental analysis through XPS indicates the presence of C, N and O (fluorine in the case of ELPs-tFF was not detected apparently due to disordered conformation and low concentration of ELPs deposited on the electrodes surface).

### Electrochemical Characterization of the ELPs Modified Electrodes

To prepare the stock 10× phosphate buffer (PBS) solution (pH 7.4), one PBS tablet was dissolved in 200 ml of DDW. Further, 5 mM ferrocyanide/ferricyanide solution redox mediator was prepared with equimolar ratio of ferrocyanide and ferricyanide in the PBS solution.

Initially, before modification, the bare electrode was analyzed electrochemically in 5 mM ferrocyanide/ferrocyanide solution in PBS using cyclic voltammetry (CV). Three cycles were performed across the potential window from −0.2 to 0.65 V (vs Ag/AgCl reference electrode), at a scan rate of 100 mV/s.

The ELPs-modified electrodes, namely the electrodes coated with the ELPs-tyr, ELPs-tFF and ELPs-pPr thin films, were tested by CV across the same potential window from −0.2 to 0.65 V at the scan rate of 100 mV/s in 10 mM PBS (pH=7.4) with the aid of 5 mM of the redox couple ferricyanide/ferrocyanide.

The voltammograms are appended in FIG. 5B. The ELPs-tyr (least hydrophobic-red) shows a slight decrease in the overall peak current followed by ELPs-tFF (hydrophobic due to —CF₃ moiety-blue), ELPs-pPr (most hydrophobic-green) exhibits the largest decrease in the peak current which indicates the formation of insulating layer inhibiting the transfer of electrons from the redox couple. As the hydrophobicity of the ELPs increased, it attained the collapsed state which covers the surface area of the electrode thereby blocking the transfer of electrons.

### Example 2

### Detection of PFOS in Buffer Solutions (100 ppm PFOS) with Different Electrochemical Techniques

Two electrochemical techniques were investigated for detection of PFOS in buffer solutions (100 ppm): cyclic voltammetry (CV) and electrochemical impedance spectroscopy (EIS).

### CV Analysis

The parameters used were as follows: initial potential [E1]=−0.2 V; final potential [E2]=0.65V; Scan rate=100 mV/sec; Number of cycles [Nc]=3). The bare gold electrode and the ELPs modified electrodes functioned as the working electrode, a commercial platinum electrode served as the counter electrode and Ag/AgCl as the reference electrode. The CV electrochemical measurements were carried out simultaneously using MuX16 integrated with Palmsens4 instrument, in 5 mM ferrocyanide/ferricyanide 10 mM PBS solutions with 100 ppm (200 μM) PFOS.

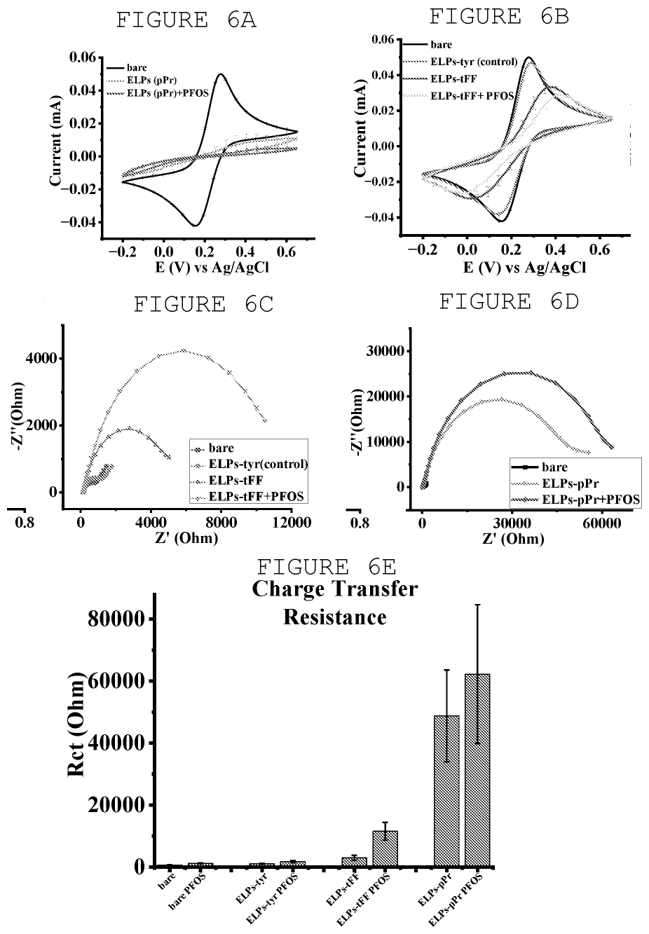

The voltammograms are shown in FIGS. 6A and 6B. The trend seen is a decrease in the peak current upon addition of PFOS to the redox solutions, for both the ELPs-tFF and ELPs-pPr electrodes (FIGS. 6A and 6B, respectively).

### EIS Analysis

EIS was also used to study the performance of various types of ELPs modified electrodes in PFOS-containing environment, i.e., with the 5 mM redox couple ferrocyanide/ferricyanide in 10 mM PBS (pH=7.4) in the presence of 100 ppm PFOS. The parameters used for EIS were as follows: Initial potential [E]=0.270 V; Initial frequency=200 kHz; final frequency=0.1 Hz; Number of points per decade=6; Sinus amplitude=25 mV. The EIS spectra were then fitted with the Randle's Circuit containing Rs=Solution Resistance, CPE=Constant Phase element (pseudo capacitor), Rct=Charge Transfer Resistance, W=Warburg element. MuX16 integrated with Palmsens4 potentiostat is used to record the EIS spectra. The electrodes were thoroughly washed with DDW and dried with nitrogen prior to the electrochemical measurement. The volume used for the analysis is 10 mL.

The results are shown in FIGS. 6C-E in the form of Nyquist plots the fluorinated ELPs (ELPs-tFF) and the hydrophobic ELPs (ELPs-pPr) (Figures C and D, respectively) and a bar chart showing the charge transfer resistance (Rct) values extracted from the EIS spectra for ELPs-tFF and ELPs-pPr (FIG. 6E). Each electrode is assigned with a pair of bars, associated with the measurement in PFOS-free solution and PFOS-containing solution). It is seen that charge transfer resistance increases in the presence of PFOS.

### Example 3

### Detection of Different Concentrations of PFOS in Buffer Solutions with Electrochemical Impedance Spectroscopy

The four electrodes (bare gold, ELPs-tyr-modified gold electrode, ELPs-tFF-modified gold electrode and ELPs-pPr-modified electrode) were investigated by EIS in solutions with PFOS concentration ranging from 500 nM-5 nM (500 nM, 400 nM, 300 nM, 200 nM, 100 nM, 5 nM) and also PFOS-free solution, in the presence of 5 mM ferrocyanide/ferricyanide acting as the redox couple. The parameters of the EIS measurement were as previously described in Example 2.

The measurements were performed in triplicates. Thereafter, the standard deviation obtained from the Y-intercept resulting from linear regression performed in Origin (Pro 2022) software is utilized in the calculation of LOD, whereas the slope of the linear regression gives the sensitivity of the sensor. All four electrodes were in contact with the test solutions at the same time, and EIS was conducted consecutively, each time one electrode acting as the working electrode.

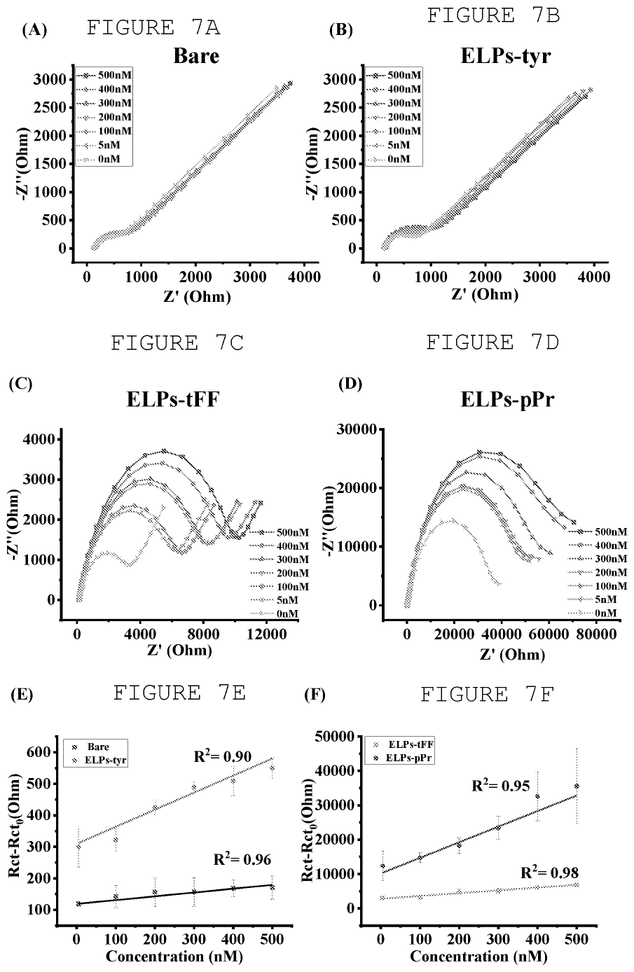

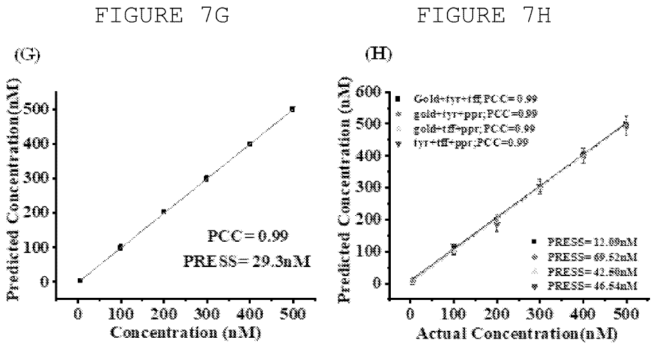

The Nyquist plots are shown in FIGS. 7A-7D for the bare electrode (A), the ELPs-tyr (control) electrode (B), the ELPs-tFF (fluorinated) electrode (C) and the ELPs-pPr (hydrophobic) electrode. It is seen that with increase in the concentration of PFOS, the diameter of the semicircle escalates, indicating an increase in the charge transfer resistance. The increment in the Rct values is a consequence of the inhibition of electron transfer from the redox mediator due to formation of an insulating film on the sensing electrode. The Rct values were well resolved. The linear regression analysis shows good co-relation for the charge transfer resistance vs PFOS concentration for different types of electrodes.

The linear regression curves for the bare and ELPs-tyr (control) electrodes are plotted in FIG. 7E, and the linear regression curves for the ELPs-tFF (fluorinated) and ELPs-pPr (hydrophobic) electrodes are plotted in FIG. 7F. The Limit of detection for the fluorinated and hydrophobic ELPs-modified electrode towards PFOS detection are 85.4±5.74 nM and 67.8±8.08 nM respectively. The sensitivity of fluorinated ELPs (ELPs-tFF) and hydrophobic ELPs (ELPs-pPr) are 8.06±0.5 Ohm/nM and 45.4±5.4 Ohm/nM respectively.

Next, PLSR model was generated in Origin (Pro 2024), combining the impedance data from all four electrodes (FIG. 7G) and from different combinations of three electrodes (FIG. 7H). The results are also arranged in a tabular form below:

TABLE A

| Electrodes combination | PCC | PRESS (nM) | LOD (nM) |
|---|---|---|---|
| 4 electrodes | 0.995 | 29.3 | 1.85 ± 0.59 |
| Gold + tyr + tff | 0.99 | 12.09 | 1.62 ± 0.51 |
| Gold + tyr + ppr | 0.99 | 69.52 | 4.5 ± 1.42 |
| Gold + tff + ppr | 0.99 | 42.50 | 6.25 ± 1.98 |
| tyr + tff + ppr | 0.99 | 46.54 | 22.8 ± 7.27 |

It is seen that the quaternary combination of electrodes and a ternary combination consisting of the bare electrode, the fluorinated electrode and the ELP-tyr electrode show very low LOD values. Specifically, the latter combination exhibited an LOD of about 1.62±0.51 nM with PCC=0.99 and PRESS=12.09 nM in buffer as per the PLSR model. The LOD values were 30 times lower with PLSR model when compared to individual regression models.

Principal Component Analysis (PCA) was performed based on Origin (Pro 2024) software, using the data from the Bode plots, namely, variation of impedance and phase change over a range of frequencies. PCA is applicable to differentiate the impedance and the phases over a range of frequency applied over its components. It was found that the differentiation of the impedance at varying concentrations is quite prominent for the fluorinated ELPs (ELPs-tFF) modified electrodes.

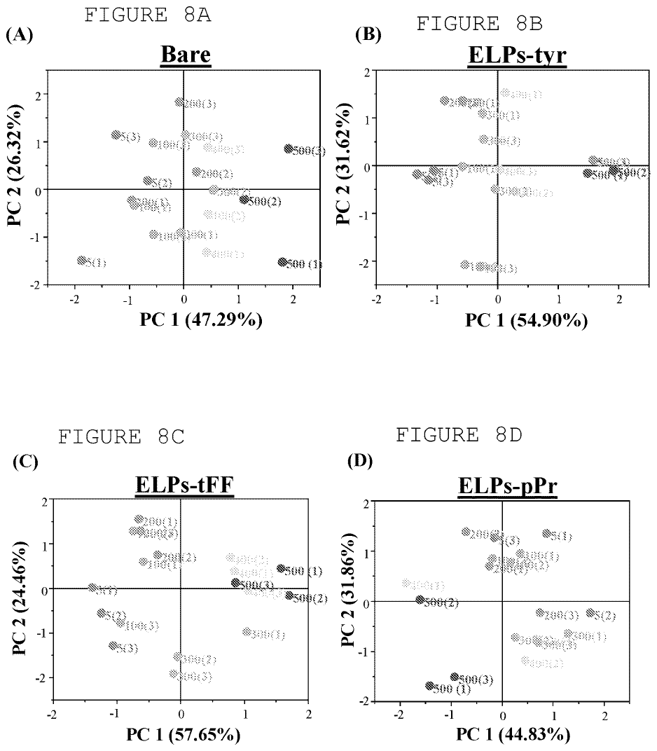

The results are shown in FIG. 8: (8A) PCA score plots for Au bare electrode; (8B) PCA score plots for ELPs-tyr (control) electrode; (8C) PCA score plots for the ELPs-tFF (fluorinated) electrode. (8D) PCA score plots for the ELPs-pPr (hydrophobic) electrode.

### Example 4

### Detection of Different Concentrations of PFOS in Simulated Groundwater Solutions with Electrochemical Impedance Spectroscopy

Simulated groundwater samples were prepared as described in Preparation 1, by adding three possible chemicals to the test solution, that could potentially interfere with PFAS analysis in real-world water systems: potassium chloride, sodium sulfate and humic acid.

EIS was conducted in the electrochemical cell described above, testing the four electrodes (bare gold, ELPs-tyr-modified gold electrode, ELPs-tFF-modified gold electrode and ELPs-pPr-modified electrode) as working electrodes in solutions with PFOS concentration ranging from 200 nM-5 nM (5 nM, 100 nM, 200 nM) and also PFOS-free solution, the presence in of 5 mM ferrocyanide/ferricyanide acting as the redox couple, and the added chemicals, in DDW. The parameters of the EIS measurement were as previously described in Example 2 (electrodes immersed in the electrochemical cup at the same time, working consecutively).

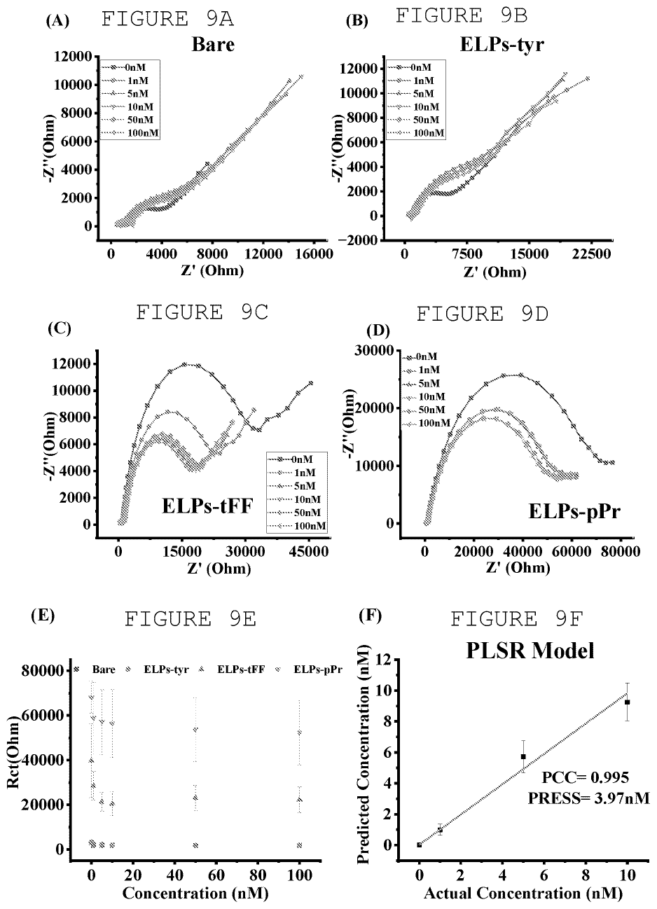

The Nyquist plots are shown in FIGS. 9A-9D for the four electrodes mentioned above, respectively. The charge transfer resistance values (Rct) were calculated from the Nyquist plots and plotted against the concentration of the PFOS in the solution, as shown collectively in FIG. 9E for all four electrodes. The results indicate a drop in charge transfer resistance values with increasing PFOS concentrations, suggesting lesser interaction between the modified electrodes and PFOS in the presence of interferents interferant.

The testing of PLSR model with the electrochemical response obtained with simulated groundwater signals at different concentrations were performed. The PFOS concentration were computed, which were LOD=140±0.057904 μm, PCC=0.995 and PRESS=3.97 nM respectively (FIG. 9F, for the four-electrode combination).

The specificity of the electrodes was evaluated in the presence humic acid (HA) as an interferant that might possibly affect the signals generated by PFOS moiety. The typical concentration range of humic acid in groundwater is from 20 μg/L to 30 mg/L. The array of electrodes was exposed to an analyte solution with and without PFOS in the presence of humic acid.

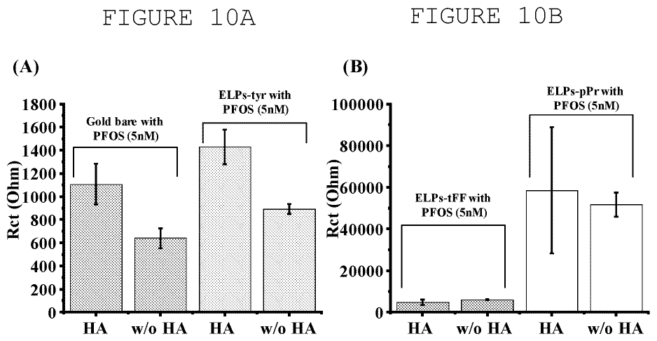

FIG. 10 shows the interferant molecule (humic acid) effect on different sensing electrodes in 5 nM PFOS solution: (A) Variation of Charge Transfer Resistance (Rct) with and without HA for bare gold and ELPS-tyr modified electrode and (B) Variation of Charge Transfer Resistance (Rct) with and without HA for ELPS-tFF and ELPS-pPr modified electrode. Statistical analysis Welch's ANOVA test was performed in the Origin Pro software. The P value was found to be greater than 0.05 thus demonstrating no significant difference with and without HA at 5 nM concentration of PFOS (the response of the fluorinated ELPs-tFF and hydrophobic ELPs-pPr coated-electrodes sensors).

### Example 5

### Detection of Different Concentrations of Spiked PFOS in Real Groundwater Solutions with Electrochemical Impedance Spectroscopy

Groundwater samples were spiked with PFOS as described in Preparation 1 and analyzed by EIS in the electrochemical cell described above, testing the four electrodes (bare gold, ELPs-tyr-modified gold electrode, ELPs-tFF-modified gold electrode and ELPs-pPr-modified electrode) as working electrodes in solutions with PFOS concentration ranging from 200 nM-5 nM (5 nM, 100 nM, 200 nM) and also PFOS-free solution, in the presence of 5 mM ferrocyanide/ferricyanide acting as the redox couple. The parameters of the EIS measurement were as previously described in Example 2.

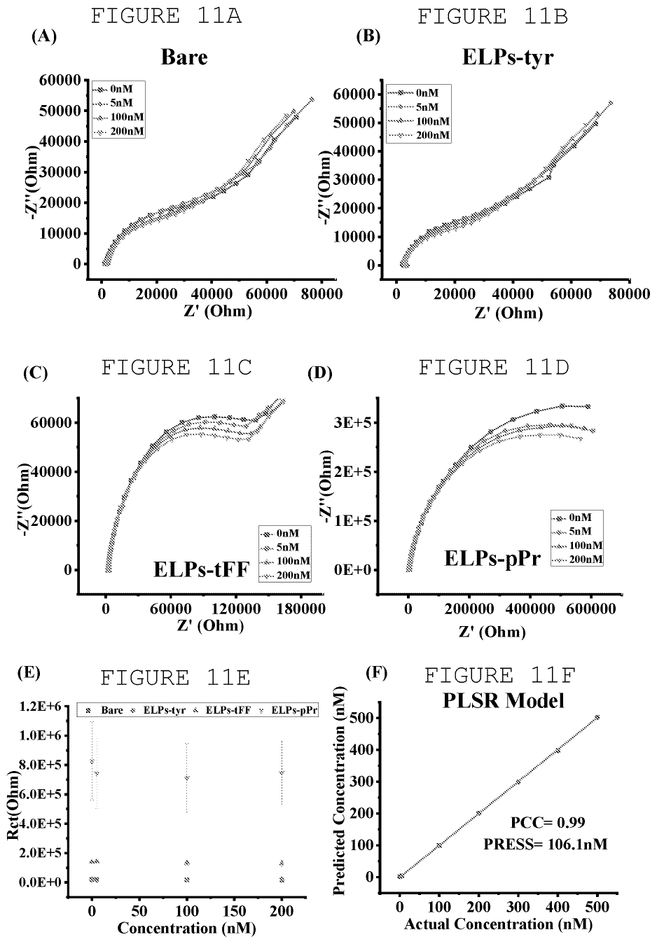

The Nyquist plots are shown in FIGS. 11A-11D for the four electrodes mentioned above, respectively. The charge transfer resistance values (Rct) were calculated from the Nyquist plots, and plotted against the concentration of the PFOS in the solution, as shown collectively in FIG. 11E for all four electrodes. The Rct values was found to decrease with the increasing concentration of PFOS, suggesting that PFOS interact with a range of molecules present in groundwater matrices, and consequently have less chance to approach the modified electrodes.

The potential of the array consisting of the four electrodes were further investigated by chemometric model, i.e., the PLSR model (FIG. 11F) to study the predict the PFOS levels in Groundwater samples. A good co-relation was observed between the predicted and the actual levels of PFOS with PCC=0.99, PRESS=106.1 nM, LOD=2.87±0.91 nM respectively. The computed values for LOD suggested the prediction from 4-1 of chemometric model the groundwater directly to quantify PFOS levels. These techniques can be implemented to calibrate and test various source of PFAS in the environmental matrices or biological samples in addition to the greater sample sizes which further help to build the accurate chemometric models to predict PFAS levels.

The following provides a description of the amino and nucleic acid sequences described herein, in the order at which they appear in the sequence listing. The sequences include three types of pentapeptide repeating units, having a variable amino acid residue “X” inserted at position 4, denoted by SEQ ID NO. 1, SEQ ID NO. 2 and SEQ ID NO. 3. They all have the amino acid sequence of VPGXG, however, they differ in the definition of the variable amino acid residue X, as detailed in the sequence listing appended herewith and shown in Table 1 below. In particular, the variable amino acid residue “X” means any natural amino acid, apart from proline, or a modified amino acid in SEQ ID NO. 1, the specific fluorinated modified amino acid residue p-Trifluoromethyl-L-phenylalanine in SEQ ID NO. 2 and the specific (hydrophobic) modified amino acid residue 4-propargyloxy-L-phenylalanine SEQ ID NO. 3. Furthermore, a repeat unit having the amino acid sequence VPGYG is denoted by SEQ ID NO. 8. In SEQ ID NO. 8, the amino acid tyrosine is present at a position that corresponds to the position of variable X in SEQ ID NO. 1.

The amino acid sequence of the non-fluorinated ELP described in the Examples is denoted by SEQ ID NO. 5, and the amino acid sequences of the ELPs described in the Examples in which p-Trifluoromethyl-L-phenylalanine (tFF) or propargyloxy-L-phenylalanine (pPr) was inserted at positions 30, 60, 90, 120, 150, 180, 210, 240, 270 and 300 of the amino acid sequence are denoted by SEQ ID NO. 6, and SEQ ID NO. 7, respectively. The nucleic acid used for encoding the polypeptides defined by SEQ ID NO. 5, SEQ ID NO. 6 and SEQ ID NO. 7 (while using the relevant specialized aminoacyl tRNA synthetases) is denoted by SEQ ID NO. 4.

TABLE 1
sequences

| SEQ ID | | |
|---|---|---|
| NO. | Sequence | Comments |
| 1 | VPGXG | X is defined as any natural |
| | | amino acid, apart from |
| | | proline, or a modified |
| | | amino acid |
| 2 | VPGXG | X is defined as p- |
| | | Trifluoromethyl-L- |
| | | phenylalanine |
| 3 | VPGXG | X is defined as 4- |
| | | propargyloxy-L- |
| | | phenylalanine |
| 4 | atggggGTTCCGGGTGGCGGcGTGCCGGGCG | Nucleic acid sequence |
| | CAGGTGTCCCGGGTGGCGGTGTGCCGGGCGC | encoding the ELPs of the |
| | AGGTGTCCCGGGTGGCGGTGTTCCGGGCtag | invention |
| | GGTGTCCCGGGTGGCGGTGTGCCGGGCGCA | |
| | GGTGTTCCGGGTGGCGGGGTGCCGGGCGCA | |
| | GGTGTTCCGGGTGGCGGTGTGCCGGGCtagG | |
| | GTGTCCCGGGTGGCGGTGTGCCGGGCGCAG | |
| | GTGTCCCGGGTGGCGGTGTTCCGGGCGCAG | |
| | GTGTCCCGGGTGGCGGTGTGCCGGGCtagGG | |
| | TGTTCCGGGTGGCGGGGTGCCGGGCGCAGG | |
| | TGTTCCGGGTGGCGGTGTGCCGGGCGCAGG | |
| | TGTCCCGGGTGGCGGTGTGCCGGGCtagGGT | |
| | GTCCCGGGTGGCGGTGTTCCGGGCGCAGGT | |
| | GTCCCGGGTGGCGGTGTGCCGGGCGCAGGT | |
| | GTTCCGGGTGGCGGGGTCCCGGGTtagGGGG | |
| | TTCCGGGTGGCGGcGTGCCGGGCGCAGGTG | |
| | TCCCGGGTGGCGGTGTGCCGGGCGCAGGTG | |
| | TCCCGGGTGGCGGTGTTCCGGGCtagGGTGT | |
| | CCCGGGTGGCGGTGTGCCGGGCGCAGGTGT | |
| | TCCGGGTGGCGGGGTGCCGGGCGCAGGTGT | |
| | TCCGGGTGGCGGTGTGCCGGGCtagGGTGTC | |
| | CCGGGTGGCGGTGTGCCGGGCGCAGGTGTC | |
| | CCGGGTGGCGGTGTTCCGGGCGCAGGTGTC | |
| | CCGGGTGGCGGTGTGCCGGGCtagGGTGTTC | |
| | CGGGTGGCGGGGTGCCGGGCGCAGGTGTTC | |
| | CGGGTGGCGGTGTGCCGGGCGCAGGTGTCC | |
| | CGGGTGGCGGTGTGCCGGGCtagGGTGTCCC | |
| | GGGTGGCGGTGTTCCGGGCGCAGGTGTCCC | |
| | GGGTGGCGGTGTGCCGGGCGCAGGTGTTCC | |
| | GGGTGGCGGGGTCCCGGGTtagGGGggtgg | |
| | ctgcTGATGA | |
| 5 | GVPGGGVPGAGVPGGGVPGAGVPGGGVPGY | Amino acid sequence of |
| | GVPGGGVPGAGVPGGGVPGAGVPGGGVPGY | the non-fluorinated ELP |
| | GVPGGGVPGAGVPGGGVPGAGVPGGGVPGY | |
| | GVPGGGVPGAGVPGGGVPGAGVPGGGVPGY | |
| | GVPGGGVPGAGVPGGGVPGAGVPGGGVPGY | |
| | GVPGGGVPGAGVPGGGVPGAGVPGGGVPGY | |
| | GVPGGGVPGAGVPGGGVPGAGVPGGGVPGY | |
| | GVPGGGVPGAGVPGGGVPGAGVPGGGVPGY | |
| | GVPGGGVPGAGVPGGGVPGAGVPGGGVPGY | |
| | GVPGGGVPGAGVPGGGVPGAGVPGGGVPGY | |
| | GGGC | |
| 6 | GVPGGGVPGAGVPGGGVPGAGVPGGGVPGX | Amino acid sequence of |
| | GVPGGGVPGAGVPGGGVPGAGVPGGGVPGX | the fluorinated ELPs, |
| | GVPGGGVPGAGVPGGGVPGAGVPGGGVPGX | X is p-Trifluoromethyl-L- |
| | GVPGGGVPGAGVPGGGVPGAGVPGGGVPGX | phenylalanine (tFF) |
| | GVPGGGVPGAGVPGGGVPGAGVPGGGVPGX | |
| | GVPGGGVPGAGVPGGGVPGAGVPGGGVPGX | |
| | GVPGGGVPGAGVPGGGVPGAGVPGGGVPGX | |
| | GVPGGGVPGAGVPGGGVPGAGVPGGGVPGX | |
| | GVPGGGVPGAGVPGGGVPGAGVPGGGVPGX | |
| | GVPGGGVPGAGVPGGGVPGAGVPGGGVPGX | |
| | GGGC | |
| 7 | GVPGGGVPGAGVPGGGVPGAGVPGGGVPGX | Amino acid sequence of |
| | GVPGGGVPGAGVPGGGVPGAGVPGGGVPGX | the hydrophobic ELPs, X |
| | GVPGGGVPGAGVPGGGVPGAGVPGGGVPGX | is 4-propargyloxy-L- |
| | GVPGGGVPGAGVPGGGVPGAGVPGGGVPGX | phenylalanine (pPr) |
| | GVPGGGVPGAGVPGGGVPGAGVPGGGVPGX | |
| | GVPGGGVPGAGVPGGGVPGAGVPGGGVPGX | |
| | GVPGGGVPGAGVPGGGVPGAGVPGGGVPGX | |
| | GVPGGGVPGAGVPGGGVPGAGVPGGGVPGX | |
| | GVPGGGVPGAGVPGGGVPGAGVPGGGVPGX | |
| | GVPGGGVPGAGVPGGGVPGAGVPGGGVPGX | |
| | GGGC | |
| 8 | VPGYG | |

1. A method for detecting and optionally quantifying one or more poly- and perfluoroalkyl substances (PFAS) in an aqueous sample, comprising adding a redox couple to the sample, electrochemically analyzing the sample with at least one surface-modified working electrode having fluorinated intrinsically disordered polypeptide deposited on its surface, by measuring current, voltage and/or calculating impedance as a PFAS-related signal, and determining the presence and optionally the level of PFAS, in the sample.

2. A method according to claim 1, wherein the sample is analyzed with an array of working electrodes comprising, alongside the fluorinated intrinsically disordered polypeptide-coated electrode, at least one working electrode selected from the group consisting of:
bare electrodes; and
surface-modified electrodes having non-fluorinated intrinsically disordered polypeptides deposited on their surface, showing different degrees of hydrophobicity; and
determining the PFAS by applying a chemometric model.

3. A method according to claim 2, wherein the fluorinated and non-fluorinated polypeptides are elastin like polypeptides (ELPs) comprising a repeat unit having the amino acid sequence VPGXG denoted by SEQ ID NO. 1, wherein X is any natural amino acid, apart from proline, or unnatural modified amino acid, characterized in that:
the fluorinated polypeptide comprises from 5 to 100% a repeat unit having the amino acid sequence VPGXG, wherein X=X¹ is a modified amino acid bearing a fluorinated functionality;
a first non-fluorinated polypeptide which comprises from 5 to 100% a repeat unit having the amino acid sequence VPGXG, wherein X=X² is a natural or unnatural amino acid bearing a hydrophilic group; and
a second non-fluorinated polypeptide which comprises from 5 to 100% a repeat unit having the amino acid sequence VPGXG, wherein X=X³ is a modified amino acid bearing a hydrophobic moiety.

4. A method according to claim 3, wherein X¹ is L-phenylalanine substituted at positions o, m, or p with a fluorinated alkyl, X² is 4-hydroxy-L-phenylalanine (tyrosine) and X³ is L-phenylalanine substituted at positions o, m, or p with an alkoxy, alkenyloxy or alkynyloxy group.

5. A method according to claim 4, wherein X¹ is trifluoromethyl-substituted L-phenylalanine and X³ is alkynyloxy-substituted L-phenylalanine.

6. A method according to claim 5, wherein X¹ and X³ are

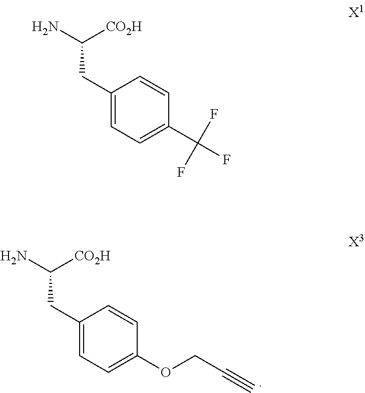 [SMILES: N[C@@H](Cc1ccc(C(F)(F)F)cc1)C(=O)O ; C#CCOc1ccc(C[C@H](N)C(=O)O)cc1]

7. A method according to claim 2, wherein:
the fluorinated polypeptide comprises the amino acid sequence shown in SEQ ID. 6, the first non-fluorinated polypeptide comprises the amino acid sequence shown in SEQ ID. 5 and the second non-fluorinated comprises the amino acid sequence in SEQ ID. 7

8. A method according to claim 2, wherein the array of working electrodes comprises:
a fluorinated hydrophobic polypeptide-coated electrode;
a bare electrode; and
at least one of the first and second non-fluorinated hydrophobic polypeptide-coated electrodes.

9. A method according to claim 1, wherein the redox couple is the ferrocyanide/ferricyanide couple.

10. A method according to claim 1, wherein the sample is analyzed by voltammetry or electrochemical impedance spectroscopy.

11. A method according to claim 10, wherein the sample is analyzed by electrochemical impedance spectroscopy (EIS).

12. A method according to claim 11, comprising analyzing the sample by EIS to generate impedance spectra, fitting the impedance spectra with Randles circuit, and calculating one or more of the following components of the Randles circuit: the charge transfer resistance, the solution resistance, the double layer capacitance and the Warburg impedance.

13. A method according to claim 12, comprising calculating the charge transfer resistance.

14. A method according to claim 11, comprising analyzing the sample by EIS to generate impedance spectra, and using a Partial Linear Regression Model (PLSR) to determine the PFA concentration based on the measured impedance spectra, wherein the PLSR model was created using as independent variable impedance values at varying frequencies, and the dependent variable is PFAS concentration in solution.

15. An electrochemical sensor comprising a working electrode with a fluorinated intrinsically disordered polypeptide, deposited on the surface of the electrode.

16. An electrochemical sensor according to claim 15, further comprising at least one working electrode selected from:
bare electrodes; and
surface-modified electrodes having non-fluorinated intrinsically disordered polypeptides deposited on their surface, showing different degrees of hydrophobicity.

17. An electrochemical sensor according to claim 16, wherein the fluorinated and non-fluorinated polypeptides are elastin like polypeptides (ELPs) comprising a repeat unit having the amino acid sequence VPGXG denoted by SEQ ID NO. 1, wherein X is any natural amino acid, apart from proline, or unnatural modified amino acid, characterized in that:
the fluorinated polypeptide comprises from 5 to 100% a repeat unit having the amino acid sequence VPGXG, wherein X=X¹ is a modified amino acid bearing a fluorinated functionality;
a first non-fluorinated polypeptide which comprises from 5 to 100%, a repeat unit having the amino acid sequence VPGXG, wherein X=X² is a natural or unnatural amino acid bearing a hydrophilic group; and
a second non-fluorinated polypeptide which comprises from 5 to 100% a repeat unit having the amino acid sequence VPGXG, wherein X=X³ is a modified amino acid bearing a hydrophobic moiety.

18. An electrochemical sensor according to claim 17, comprising: a fluorinated intrinsically disordered polypeptide-coated working electrode;
a bare electrode; and
at least one of the first and second non-fluorinated hydrophobic polypeptide-coated electrodes.

19. An electrochemical sensor according to claim 15, wherein the electrodes are deployed on a substrate, optionally atop of a titanium adhesion layer attached to the substrate, the electrodes are disc-shaped, and are spaced apart from each other, the electrodes are protruding from the surface of the substrate, wherein each electrode is encircled by a wall made of an epoxy polymer and is joined by a thin wire to a contact pad.

20. An electrochemical sensor according to claim 15, further comprising a counter electrode and a reference electrode.

21.-25. (canceled)


SEQUENCE LISTING (8 sequences)

SEQ ID NO: 1 - protein, 5 aa, synthetic construct
  VARIANT 4: note = X is any natural amino acid, apart from proline, or a modified amino acid
VPGXG

SEQ ID NO: 2 - protein, 5 aa, synthetic construct
  SITE 4: note = p-Trifluoromethyl-L-phenylalanine
VPGXG

SEQ ID NO: 3 - protein, 5 aa, synthetic construct
  SITE 4: note = 4-propargyloxy-L-phenylalanine
VPGXG

SEQ ID NO: 4 - DNA, 921 bp, synthetic construct
  source 1..921: mol_type = other DNA
atgggggttccgggtggcggcgtgccgggcgcaggtgtcccgggtggcggtgtgccgggcgcaggtgtcccgggtggcggtgttccgggctagggtgtcccgggtggcggtgtgccgggcgcaggtgttccgggtggcggggtgccgggcgcaggtgttccgggtggcggtgtgccgggctagggtgtcccgggtggcggtgtgccgggcgcaggtgtcccgggtggcggtgttccgggcgcaggtgtcccgggtggcggtgtgccgggctagggtgttccgggtggcggggtgccgggcgcaggtgttccgggtggcggtgtgccgggcgcaggtgtcccgggtggcggtgtgccgggctagggtgtcccgggtggcggtgttccgggcgcaggtgtcccgggtggcggtgtgccgggcgcaggtgttccgggtggcggggtcccgggttagggggttccgggtggcggcgtgccgggcgcaggtgtcccgggtggcggtgtgccgggcgcaggtgtcccgggtggcggtgttccgggctagggtgtcccgggtggcggtgtgccgggcgcaggtgttccgggtggcggggtgccgggcgcaggtgttccgggtggcggtgtgccgggctagggtgtcccgggtggcggtgtgccgggcgcaggtgtcccgggtggcggtgttccgggcgcaggtgtcccgggtggcggtgtgccgggctagggtgttccgggtggcggggtgccgggcgcaggtgttccgggtggcggtgtgccgggcgcaggtgtcccgggtggcggtgtgccgggctagggtgtcccgggtggcggtgttccgggcgcaggtgtcccgggtggcggtgtgccgggcgcaggtgttccgggtggcggggtcccgggttaggggggtggctgctgatga

SEQ ID NO: 5 - protein, 304 aa, synthetic construct
GVPGGGVPGAGVPGGGVPGAGVPGGGVPGYGVPGGGVPGAGVPGGGVPGAGVPGGGVPGYGVPGGGVPGAGVPGGGVPGAGVPGGGVPGYGVPGGGVPGAGVPGGGVPGAGVPGGGVPGYGVPGGGVPGAGVPGGGVPGAGVPGGGVPGYGVPGGGVPGAGVPGGGVPGAGVPGGGVPGYGVPGGGVPGAGVPGGGVPGAGVPGGGVPGYGVPGGGVPGAGVPGGGVPGAGVPGGGVPGYGVPGGGVPGAGVPGGGVPGAGVPGGGVPGYGVPGGGVPGAGVPGGGVPGAGVPGGGVPGYGGGC

SEQ ID NO: 6 - protein, 304 aa, synthetic construct
  SITE 30: note = p-Trifluoromethyl-L-phenylalanine
  SITE 60: note = p-Trifluoromethyl-L-phenylalanine
  SITE 90: note = p-Trifluoromethyl-L-phenylalanine
  SITE 120: note = p-Trifluoromethyl-L-phenylalanine
  SITE 150: note = p-Trifluoromethyl-L-phenylalanine
  SITE 180: note = p-Trifluoromethyl-L-phenylalanine
  SITE 210: note = p-Trifluoromethyl-L-phenylalanine
  SITE 240: note = p-Trifluoromethyl-L-phenylalanine
  SITE 270: note = p-Trifluoromethyl-L-phenylalanine
  SITE 300: note = p-Trifluoromethyl-L-phenylalanine
GVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGVPGGGVPGAGVPGGGVPGAGVPGGGVPGXGGGC

SEQ ID NO: 7 - protein, 304 aa, synthetic construct; identical to SEQ ID NO: 6
  SITE 30: note = 4-propargyloxy-L-phenylalanine
  SITE 60: note = 4-propargyloxy-L-phenylalanine
  SITE 90: note = 4-propargyloxy-L-phenylalanine
  SITE 120: note = 4-propargyloxy-L-phenylalanine
  SITE 150: note = 4-propargyloxy-L-phenylalanine
  SITE 180: note = 4-propargyloxy-L-phenylalanine
  SITE 210: note = 4-propargyloxy-L-phenylalanine
  SITE 240: note = 4-propargyloxy-L-phenylalanine
  SITE 270: note = 4-propargyloxy-L-phenylalanine
  SITE 300: note = 4-propargyloxy-L-phenylalanine

SEQ ID NO: 8 - protein, 5 aa, synthetic construct
VPGYG